(19) United States
(12) Patent Application Publication
(10) Pub. No.: US 2025/0177600 A1
(43) Pub. Date: Jun. 5, 2025

(54) SYSTEM TO PURIFY AND DEODORIZE FLUIDS AND ASSOCIATED DEVICES AND METHODS

(71) Applicant: Green-Ox, LLC, Burnsville, MN (US)

(72) Inventors: Russell Paskewitz, Lakeville, MN (US); Scott Drever, Ankeny, IA (US); Mark Mark Wootten, Ankeny, IA (US)

(21) Appl. No.: 18/962,680

(22) Filed: Nov. 27, 2024

Related U.S. Application Data
(60) Provisional application No. 63/604,522, filed on Nov. 30, 2023.

(51) Int. Cl. A61L 9/20

(52) U.S. Cl. CPC A61L 9/20; A61L 2209/111; A61L 2209/12; A61L 2209/212

(57) ABSTRACT

The disclosed systems, devices, and methods relate to a fluid purification system comprising a housing open at each end through which a fluid is able to pass; and a cassette within the housing comprising a plurality of lamps capable of emitting light, wherein the cassette is removable from the housing, such as for ease of maintenance.

### CROSS-REFERENCE TO RELATED APPLICATION(S)

This application claims the benefit under 35 U.S.C. § 119 (e) to U.S. Provisional Application 63/604,522, filed Nov. 30, 2023, and entitled “SYSTEM TO PURIFY AND DEODORIZE FLUIDS AND ASSOCIATED DEVICES AND METHODS”, which is hereby incorporated herein by reference in its entirety for all purposes.

### TECHNICAL FIELD

The disclosure relates to systems, devices, and methods for purifying fluids. More specifically, this disclosure relates to systems, devices, and methods for removing or degrading contaminants from fluid streams such as flue gas streams and industrial exhaust streams.

### BACKGROUND

Air purification is important in industrial facilities where the emission of airborne pollutants may have a significant impact on the environment, health, and safety of employees and neighboring communities. Traditional air purification methods, such as mechanical filtration and chemical scrubbers, have been utilized to control the release of particulate matter, volatile organic compounds, and other harmful substances from industrial processes. However, these methods often suffer from limitations in efficiency, maintenance requirements, and environmental footprint.

Industrial businesses require air purification systems that may operate efficiently and reliably in harsh conditions in order to neutralize a broad spectrum of contaminants. Furthermore, air purification systems must adhere to rigorous safety standards.

### BRIEF SUMMARY

Described herein are various systems, devices, and methods for purifying and deodorizing various fluids, such as contaminated air. Although various the implementations described herein are described with air being the fluid to be used with the system, this is in no way intended to be restrictive and other fluids may be used with the system, as would be understood by those of skill in the art. Many other fluids, such as flue gas, petrochemical gases, steam, and similar fluids could be purified without departing from the scope of this disclosure.

Disclosed herein are systems, devices, and methods for purifying and deodorizing while also providing for increased ease of maintenance, increased containment of potentially harmful intermediates, and increased scalability to treat larger volumes of contaminated fluids.

Example 1 relates to a fluid purification system comprising a housing open at each end through which a fluid is able to pass, and a cassette within the housing comprising a plurality of lamps capable of emitting light, wherein the cassette is removable from the housing.

Example 2 relates to Examples 1 and 3-7, further comprising a plurality of ballasts capable of supplying electricity to the plurality of lamps.

Example 3 relates to Examples 1-2 and 4-7, wherein the lamps emit light in the ultraviolet wavelength band.

Example 4 relates to Examples 1-3 and 5-7, wherein the light in the ultraviolet wavelength band breaks down contaminants in the fluid.

Example 5 relates to Examples 1-4 and 6-7, wherein the light in the ultraviolet wavelength band generates ozone in the fluid.

Example 6 relates to Examples 1-5 and 7, further comprising an ozone monitor capable of detecting an ozone amount in the fluid, wherein the plurality of ballasts and plurality of lamps are configured to vary the amount of ultraviolet light emitted based on the ozone amount detected.

Example 7 relates to Examples 1-6, wherein a detected ozone amount in the fluid below a lower threshold causes the plurality of ballasts and plurality of lamps to increase the amount of ultraviolet light emitted and a detected ozone amount in the fluid above an upper threshold causes the plurality of ballasts and plurality of lamps to decrease the amount of ultraviolet light emitted.

Example 8 relates to a fluid purification system comprising a housing with a first end and a second end through which a fluid is able to pass from the first end to the second end, a cassette within the housing comprising a plurality of lamps capable of spurring the generation of ozone, a backflow damper disposed within the housing at the first end of the housing capable of allowing, restricting, and stopping the passage of fluid through the housing, and a makeup air damper disposed proximal to the first end of the housing capable of allowing, restricting, and stopping the passage of fluid through the housing, wherein the cassette is removable from the housing.

Example 9 relates to Examples 8 and 10-14, further comprising a plurality of ballasts capable of supplying electricity to the plurality of lamps.

Example 10 relates to Examples 8-9 and 11-14, further comprising an ozone monitor capable of detecting an ozone amount in the fluid.

Example 11 relates to Examples 8-10 and 12-14, further comprising an airflow sensor capable of measuring the flow of the fluid though the housing.

Example 12 relates to Examples 8-11 and 13-14, wherein the backflow damper is configured to close based upon the measured fluid flow by the airflow sensor.

Example 13 relates to Examples 8-12 and 14, further comprising a temperature probe positioned in the housing to measure the temperature of the fluid

Example 14 relates to Examples 8-13, wherein the makeup air damper is configured to adjust its position based on the temperature of the fluid.

Example 15 relates to a fluid purification system comprising a housing with a first end and a second end wherein a fluid is able to pass from the first end to the second end, a cassette within the housing comprising a plurality of lamps capable of spurring the generation of ozone, and a flow restrictor within the housing positioned between the cassette and the second end of the housing, wherein the cassette is removable from the housing, and wherein the flow restrictor increases the time needed for fluid to pass through the housing.

Example 16 relates to Examples 15 and 17-20, wherein the flow restrictor is one or more adjustable flow promoters.

Example 17 relates to Examples 15-16 and 18-20, wherein the flow restrictor is a plurality of baffles.

Example 18 relates to Examples 15-17 and 19-20, wherein the lamps emit light in the ultraviolet wavelength band.

Example 19 relates to Examples 15-18 and 20, further comprising an ozone monitor capable of detecting ozone in the fluid.

Example 20 relates to Examples 15-19, wherein the lamps are configured to emit more or less ultraviolet wavelength light based on the ozone detected in the fluid.

While multiple embodiments are disclosed, still other embodiments of the disclosure will become apparent to those skilled in the art from the following detailed description, which shows and describes illustrative embodiments of the disclosed system, devices, and methods. As will be realized, the disclosed system, devices, and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a diagram of the system, according to one implementation.

FIG. 2 is a flow diagram of the system, according to one implementation.

FIG. 3 is a close-up view of the housing with the inlet duct and backflow damper removed, according to one implementation.

FIG. 4 is an isometric view of a single housing with cassettes inserted, according to one implementation.

FIG. 5 is an isometric view of the device where the cassettes are partially removed from the housing through the cabinet, according to one implementation.

FIG. 6 is an isometric view of the device where the cabinet doors are open and the cassettes are inserted, according to one implementation.

FIG. 7 is an isometric view of a remote electrical cabinet used in the system, according to one implementation.

FIG. 8A is a front view of a remote electrical cabinet used in the system, according to one implementation.

FIG. 8B is a side view of a remote electrical cabinet used in the system, according to one implementation.

FIG. 9 is a view of a cassette removed from the housing, according to one implementation.

FIG. 10 is an isometric view of a single housing with cassettes removed, according to one implementation.

FIG. 11 is an isometric view of the system, according to one implementation.

FIG. 12 is a cross-sectional view of the system, according to one implementation.

FIG. 13 is an isometric view of the system with one housing, cassettes inserted, and doors closed, according to one implementation.

FIG. 14 is an isometric view of the system with cassettes removed and doors open, according to one implementation.

FIG. 15A is a depiction of the system on a trailer, according to one implementation.

FIG. 15B is an end-on view of the system showing the cabinet and primary reaction chamber, according to one implementation.

FIG. 16 is a cross-sectional view of the system, shown from the top with the access door open, according to one implementation.

FIG. 17 is a cross-sectional side view of the system, according to one implementation.

FIG. 18 is a cross-sectional top view of the system with the access door closed, according to one implementation.

FIG. 19 shows an adjustable flow promoter, according to one implementation.

FIG. 20 shows the system with the perforated plate and primary reaction chamber visible, according to one implementation.

FIG. 21 is a cut-away view of the system, according to one implementation.

FIG. 22 shows the system with the perforated plate partially removed, according to one implementation.

FIG. 23 is a cut-away view of the tempering chamber, according to one implementation.

FIG. 24 is an end-on view of the system with the housing doors open, according to one implementation.

FIG. 25A is an isometric view of the system, according to one implementation.

FIG. 25B is a side view of the system, according to one implementation.

FIG. 26 is a side view of the system with two housings being used in tandem, according to one implementation.

FIG. 27A is a flow velocity diagram of the system in use without flow restrictors, according to one implementation.

FIG. 27B is a flow velocity diagram of the system in use with two adjustable flow promoters, according to one implementation.

FIG. 27C is a scale used for quantifying the diagrams shown in FIGS. 27A and 27B.

### DETAILED DESCRIPTION

The various implementations disclosed and contemplated herein relate to various devices, systems, and methods for purifying and deodorizing air. Various implementations use light with wavelengths in or around the ultraviolet spectrum (UV), optionally UV-C, to directly and indirectly break down air impurities by forming ozone, which breaks down the air impurities by oxidizing the molecules, as would be generally understood. Various implementations may contain a variety of process control and safety features to ensure efficient and safe operation of the system, such as temperature monitoring, ozone containment systems, airflow monitoring, and active interlocks.

The system described herein may be used for air purification applications and the like as would be appreciated by those of skill in the art in light of this disclosure. Various implementations include the use of the system in purifying and deodorizing the air of industrial cooking operations, restaurant kitchens, rendering plants, waste incineration facilities, fermentation facilities, and various other facilities that produce unpleasant volatile compounds. Various additional applications are possible and contemplated herein.

Various industries emit or produce fluids with contaminants that may be harmful to people, animals, and the environment and/or may carry undesirable smells. Examples of harmful chemicals emitted or produced in these fluid streams include nitrogen oxides, carbon monoxide, carbon dioxide, sulfur oxides, methane, and ammonia, among others, which are common in flue gas streams from burn facilities and petrochemical facilities. Examples of contaminants with undesirable smells include polycyclic aromatic hydrocarbons, nitrated polycyclic aromatic hydrocarbons, aldehydes, and aromatic amines, among others, which are often found as gaseous or vaporous emissions of industrial frying operations.

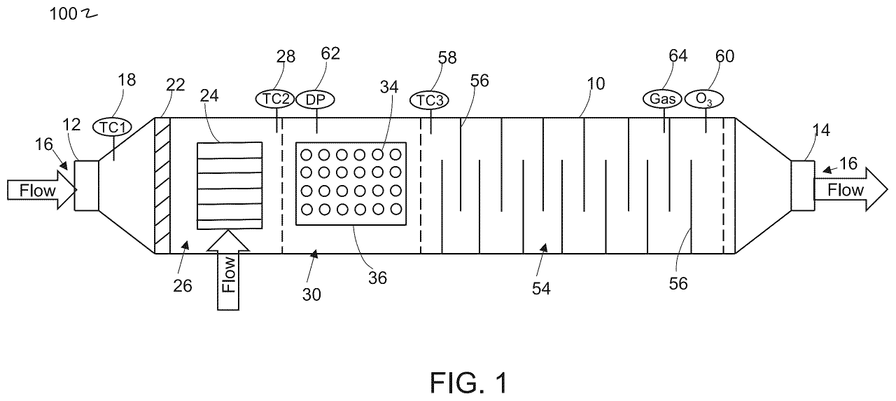

FIG. 1 shows a schematic diagram of one implementation of the system 100. In some implementations, the system 100 includes a housing 10. In various implementations, the housing 10 is elongate. The housing 10 may be a square duct but may also be rectangular, round, or any other geometry known to those of skill in the art.

In various implementations, the system 100 has an inlet duct 12 and an outlet duct 14, on opposite ends of the housing 10. The inlet duct 12 and outlet duct 14 are open such that air or other fluids may flow through the inlet duct 12 into the lumen of the housing 10 and out of the outlet duct 14. Fluid flows from the inlet duct 12 to the outlet duct 14 via a channel 16 through the housing 10. In certain implementations, the inlet duct 12 and outlet duct 14 have tapered sections that adjust diameter of the channel 16, when the diameter of the inlet duct 12, outlet duct 14, and the housing 10 are different.

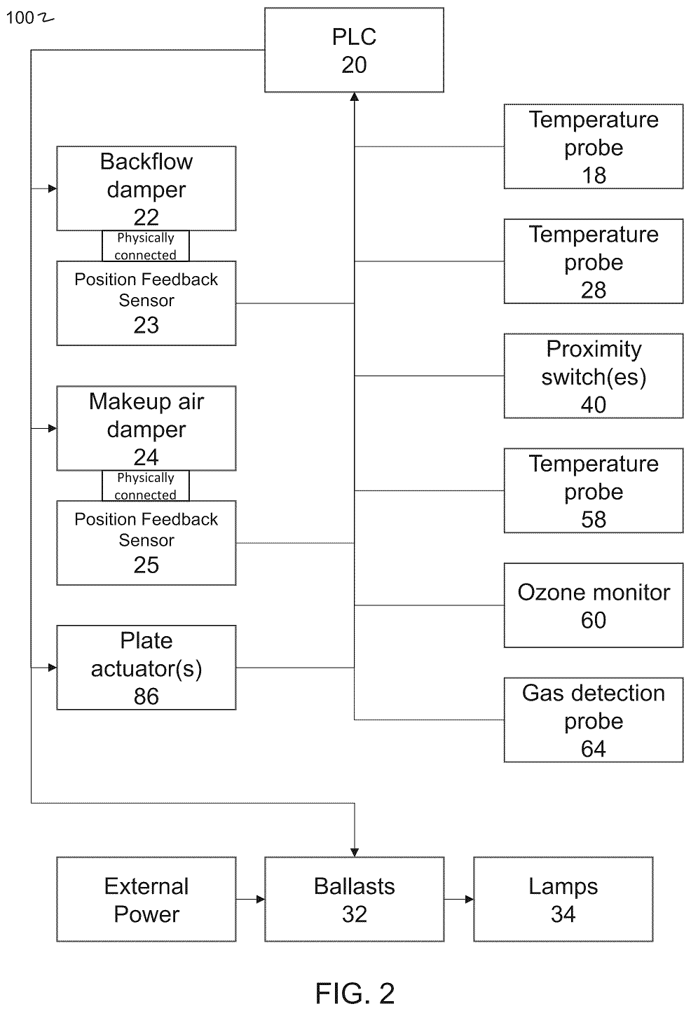

In certain implementations, near the inlet duct 12, the system 100 includes a temperature probe 18. In various implementations, the temperature probe 18 may be a thermocouple, resistance temperature detector (RTD), thermistor, semiconductor based integrated circuit (IC), or any other device for measuring temperature known in the art. Optionally, the temperature probe 18 is mounted with the sensing end inserted through the inlet duct 12 or housing 10 such that it may measure the temperature of the fluid in the channel 16, but with other components, such as electronic components, mounted outside of the housing 10 or inlet duct 12. In some implementations, a thermowell may be mounted in the inlet duct 12, which allows the temperature probe 18 to measure the temperature of the fluid in the channel 16 without being in direct contact with the fluid. As shown in FIG. 2, the temperature probe 18 may be in electronic communication with a programmable logic controller (PLC) 20. The PLC 20 may be configured to receive various inputs including digital, analog, or other electronic signals, such as from the temperature probe 18, make algorithmic decisions based on those inputs, and send various outputs in the form of digital, analog, or other electronic signals. Alternative locations and configurations for the temperature probe 18 are possible and would be understood.

Returning to FIG. 1, inside of the housing 10, optionally near the end closest to the inlet duct 12, may be a backflow damper 22. In various implementations, the backflow damper 22 allows fluid flow, such as air flow, across it when open, but does not allow such flow when closed. In certain implementations, the backflow damper 22 takes the form of an actuated damper, and may be opened or closed automatically, such as by using a motorized or pneumatic actuator. In various further implementations, the backflow damper 22 may also take the form of a multi-blade damper, a single blade damper, and inlet vane damper, or any other damper style appreciated in the art.

In various implementations, the opening or closing of the backflow damper 22 is done by an output signal sent from the PLC 20; the control may be manual, automatic, or semiautomatic. In some implementations, the backflow damper 22 has a position feedback sensor 23, shown in FIG. 2, that allows for detection of the position of the backflow damper 22 by the PLC 20. In some implementations, the position feedback sensor 23 transmits a signal indicating open/not-open, closed/not-closed, or open/closed. In other implementations, the position feedback sensor 23 transmits a signal that indicates the percent open or percent closed of the backflow damper 22. The signals from the feedback sensor 23 may be digital or analog.

As would be understood, dust buildup, corrosion, loss of utilities, and other outside factors may compromise the ability for the backflow damper 22 to actuate despite an output signal being sent from the PLC 20 to adjust the position of the backflow damper 22, and as such a position feedback sensor 23 signal may indicate the actual position of the backflow damper 22 in real-time or near real-time. In various implementations, the backflow damper 22 is in electronic communication with the PLC 20 for one or more of the purposes of actuation and position feedback.

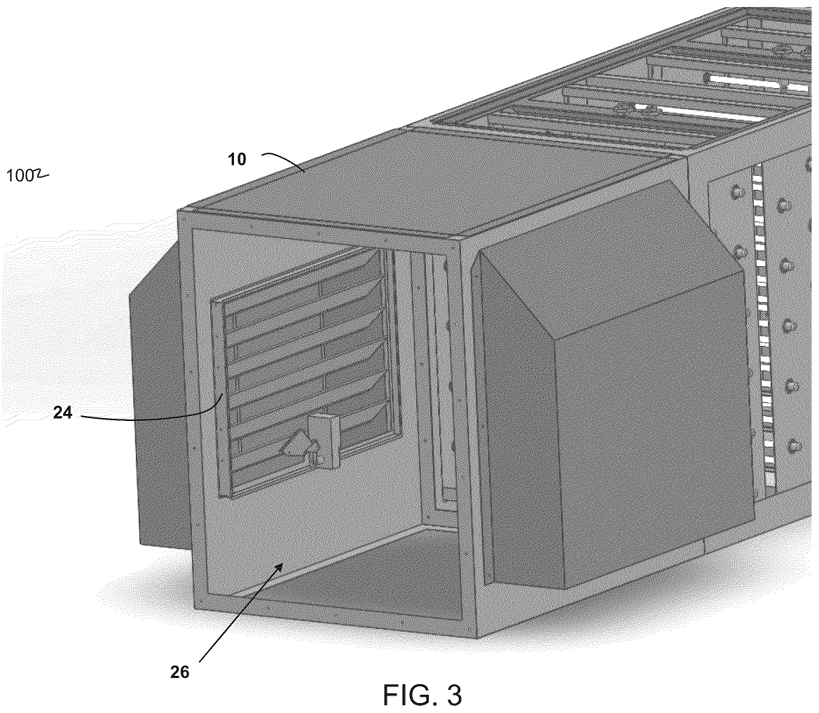

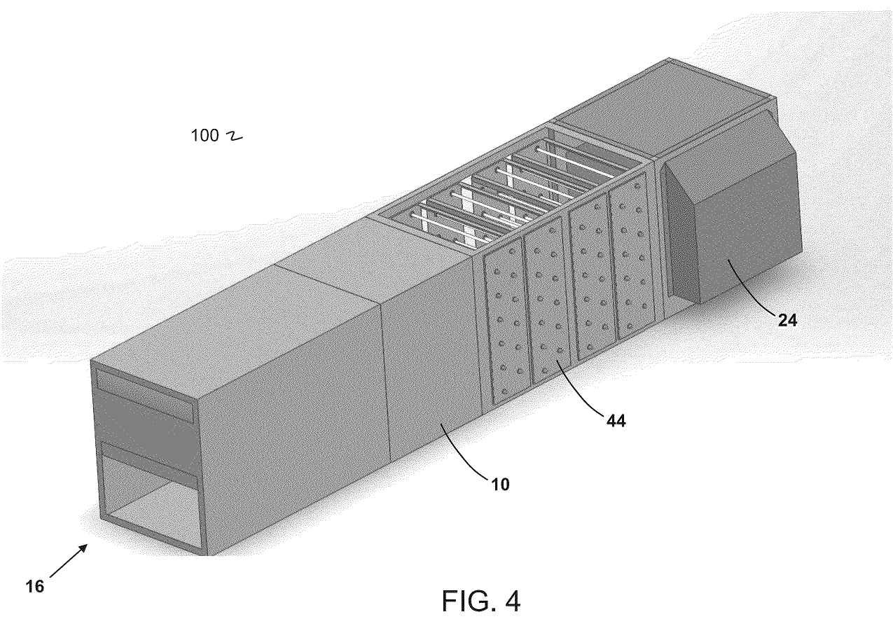

Turning to FIGS. 3 and 4, in some implementations, there is one or more makeup air dampers 24 in the housing 10 near the backflow damper 22. The makeup air damper 24 allows fluid flow, such as air flow, across it when open, but does not allow such flow when closed. In various implementations, the makeup air damper 24 is an actuated damper, and may be opened or closed automatically, such as by the use of a motorized or pneumatic actuator. In various further implementations, the makeup air damper 24 may also take the form of a multi-blade damper, a single blade damper, and inlet vane damper, or any other damper style appreciated in the art.

Returning to FIG. 2 and as would be appreciated, the opening or closing of the makeup air damper 24 is done by an output signal sent from the PLC 20. In some implementations, the makeup air damper 24 has a position feedback sensor 25 that allows for detection of the position of the makeup air damper 24 by the PLC 20. In some implementations, the position feedback sensor 25 may transmit a digital signal indicating open/not-open, closed/not-closed, or open/closed. In other implementations, the position feedback sensor 25 may transmit an analog signal indicating the percent open or percent closed of the makeup air damper 24.

As dust buildup, corrosion, loss of utilities, and other outside factors compromise the ability for the makeup air damper 24 to actuate despite an output signal being sent from the PLC 20 to adjust the position of the makeup air damper 24, the position feedback sensor 25 may provide an actual position of the makeup air damper 24 in real-time or near real-time. In various implementations, the makeup air damper 24 is in electronic communication with the PLC 20 for one or more of the purposes of actuation and position feedback.

Turning to FIG. 1, the backflow damper 22 and makeup air damper 24 may optionally be positioned such that fluid may flow through either or both into a tempering chamber 26. The backflow damper 22 may be positioned to allow warm and contaminated fluid into the tempering chamber 26. The makeup air damper 24 may be positioned to allow cooler fluid into the tempering chamber 26. The tempering chamber 26 is a section within the housing 10 that allows the air streams entering through the backflow damper 22 and makeup air damper 24 to mix. This mixing of air streams alters the temperature of the air within the tempering chamber 26. The tempering chamber 26, shown without the backflow damper 22, may be seen in FIG. 3.

Still in FIG. 1, in the tempering chamber 26, there may be a temperature probe 28. In various implementations, the temperature probe 28 may be a thermocouple, RTD, thermistor, IC, or any other device for measuring temperature known in the art. The temperature probe 28 may be mounted with the sensing end inserted through the housing 10 so it may measure the temperature of any fluid, typically air, in the channel 16 but with other components, such as electronic components, mounted outside of the housing 10. In some implementations, a thermowell may be mounted in the housing 10, which allows the temperature probe 28 to measure the temperature of the fluid in the channel 16 without being in direct contact with the fluid. As shown in FIG. 2, the temperature probe 28 may be in electronic communication with a PLC 20. As is discussed variously herein, the PLC 20 is configured to receive digital, analog, or other electronic signals, such as from the temperature probe 28, make algorithmic decisions based on those inputs, and send various outputs in the form of digital, analog, or other electronic signals. Alternative locations and configurations for the temperature probe 28 are possible and would be understood.

In various implementations, the PLC 20 will monitor the temperature of the tempering chamber 26 through the signal received from the temperature probe 28 and send output signals to actuate the makeup air damper 24 and/or the backflow damper 22 based on the temperature of the tempering chamber 26. In some implementations, the PLC 20 will be programmed to keep the tempering chamber 26 at a set temperature, optionally about 120° F. In certain implementations, the temperature in the tempering chamber 26 is modulated by the PLC 20 sending an output signal to the makeup air damper 24 and/or backflow damper 22 to open or close to adjust air flow as necessary. In a more specific example, when the temperature of the tempering chamber 26 is above the set temperature (optionally about 120° F.) the PLC 20 signal the makeup air damper 24 to become more open to draw in more cool air to decrease the temperature. Additionally or alternatively, the PLC 20 may signal the backflow damper 22 to become more closed to decrease the amount of warm air entering the tempering chamber 26 thereby reducing the temperature.

Continuing with this specific example, when the temperature of the tempering chamber 26 is below the set temperature (optionally about 120° F.) the PLC 20 may send a signal to the makeup air damper 24 to become more closed/restrict air flow and/or to the backflow air damper to become more open/allow increased air flow to increase temperature in the tempering chamber 26.

In another example, if the position feedback of the makeup air damper 24 indicates it is completely open or otherwise allowing the maximum amount of air into the tempering chamber 26, but the temperature of the tempering chamber 26 continues to remain above the set temperature (optionally about 120° F.), the PLC 20 may send an output signal to the backflow damper 22 instructing it to close, either completely or partially.

It would be understood in the art that the set temperature (optionally 120° F.) may be selected to reduce thermal degradation of components of the system 100, and that various target temperatures could be selected, depending on component temperature ratings.

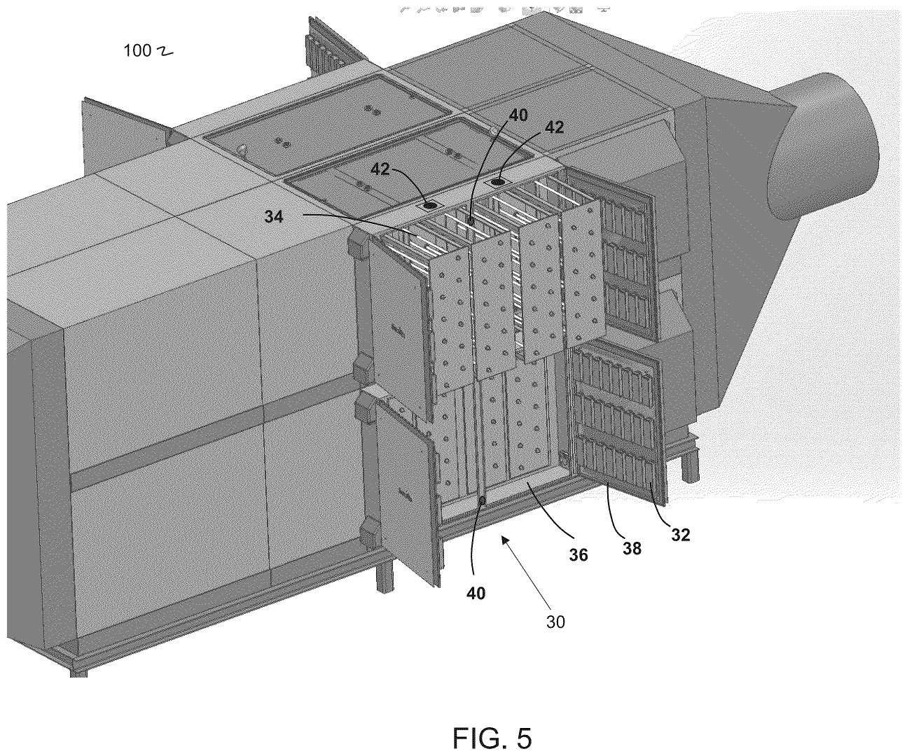

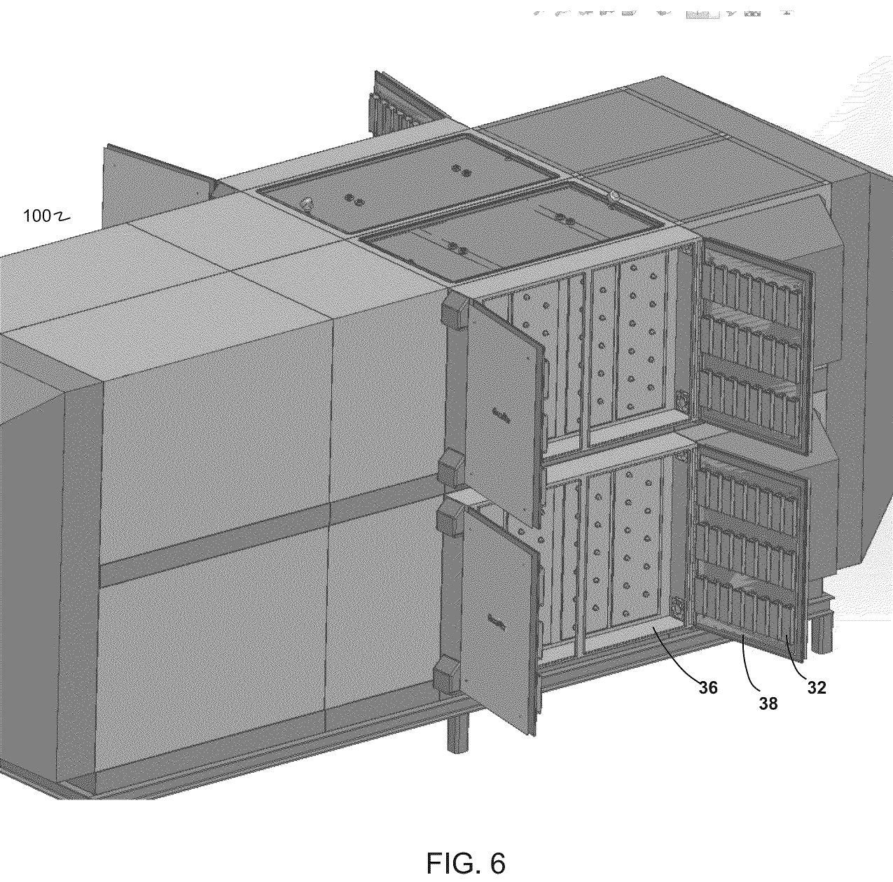

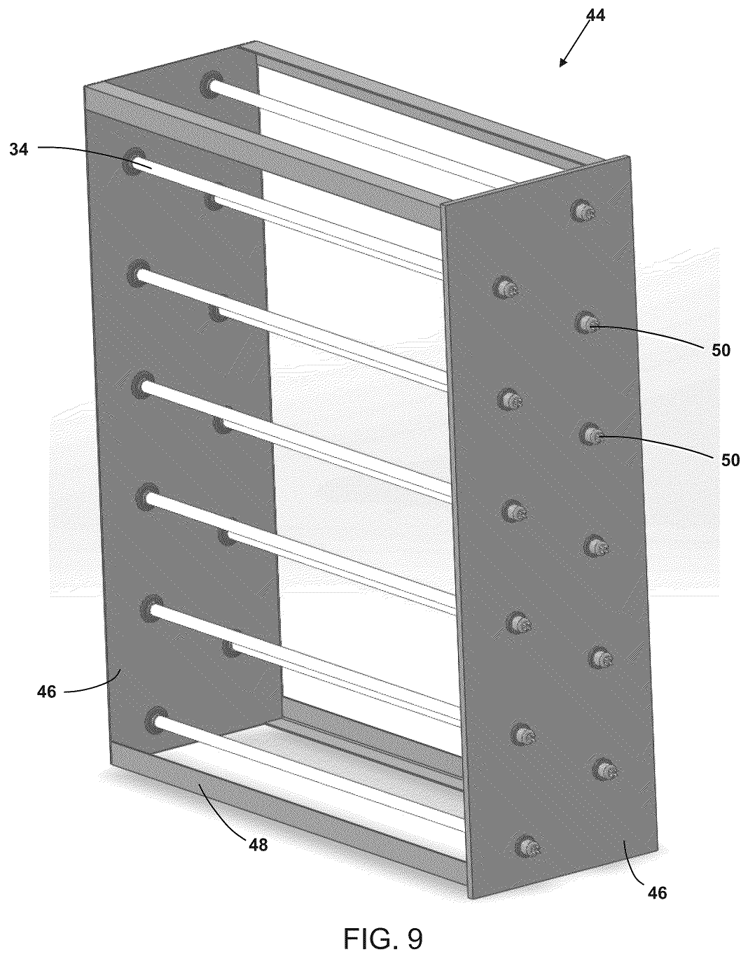

In various implementations, a primary reaction chamber 30 is within the housing 10 and optionally proximal to the tempering chamber 26. The primary reaction chamber 30 may have a plurality of ballasts 32 (shown best in FIGS. 5 and 6) and a plurality of lamps 34 (shown best in FIG. 9).

In some implementations, such as are shown in FIGS. 5 and 6, the ballasts 32 may be mounted in a cabinet 36. Shown in FIG. 2, the ballasts 32 may be electrically connected to the lamps 34 and send electrical power to the lamps 34. The ballasts 32 may be configured to limit and adjust the amount of electrical power sent to the lamps 34. The limits and adjustments made to the electrical power by the ballasts 32 may be made for current, voltage, and/or any other measure of electrical output. The amount of electrical power sent from the ballasts 32 to the lamps 34 is optionally controlled by an output signal from the PLC to the ballasts 32. In certain implementations, the ballasts 32 are remote from the housing 10.

Returning to FIG. 5, in various implementations, the lamps 34 are mounted within the primary reaction chamber 30. The lamps 34 may be configured to emit light within any wavelength band, as may be desired for any particular implementation. The lamps 34 may, in certain implementations, emit ultraviolet (UV) light.

In some implementations, the cabinet 36 has doors 38 that allow for access, such as for maintenance. Shown in FIGS. 2 and 5, the doors 38 on the cabinet 36 may have proximity switches 40, that may be in electronic communication with the PLC 20. The PLC 20 may be configured to not send an output signal to the ballasts 32 if the proximity switches 40 are disengaged, i.e. the doors 38 on the cabinet 36 are open. The proximity switches 40 may also be active interlocks that do not allow for the doors 38 to be opened until the PLC 20 disengages the active interlock mechanism through an electronic output signal.

Still in FIG. 5, the cabinet 36 may possess one or more cabinet coolers 42 which regulate the temperature of the cabinet 36 by either exhausting relatively hot air in the cabinet 36, by injecting relatively cool air from the surrounding environment into the cabinet 36, or by simultaneously exhausting relatively hot air and injecting relatively cool air. In some implementations, the cabinet coolers 42 are an air conditioning system designed for electrical cabinets. In various implementations, the cabinet coolers 42 possess an internal temperature probe that allows the cabinet coolers 42 to target a specific cabinet 36 operating temperature.

In one specific example, the target cabinet 36 operating temperature is about 105° F. or below. It would be understood in the art that the temperature 105° F. would be selected to reduce thermal degradation of components of the system 100 in the cabinet 36, and that various other target temperatures could be selected, depending on component temperature ratings or other variables as would be understood.

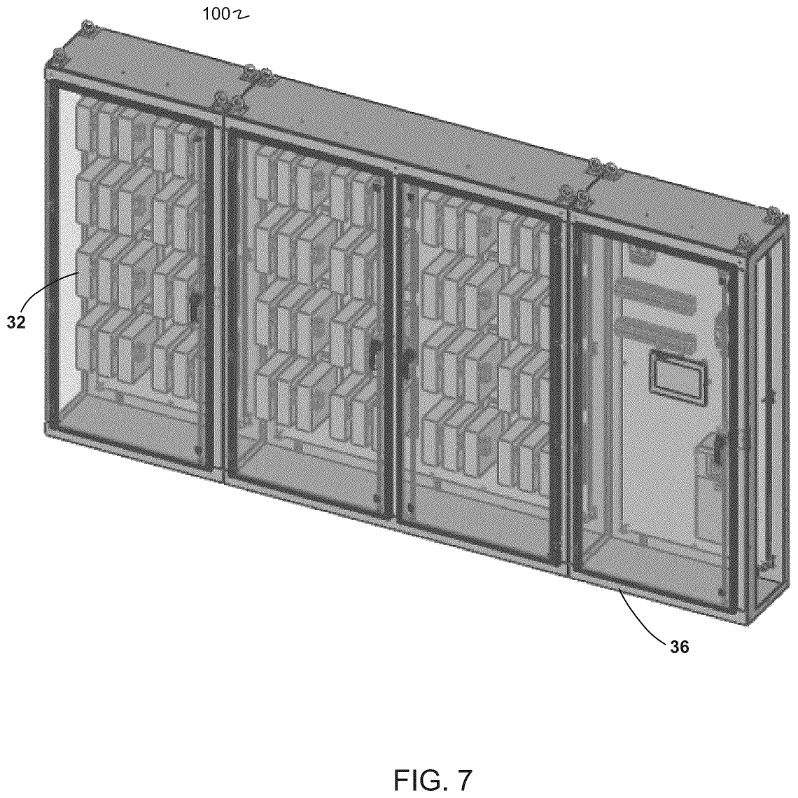

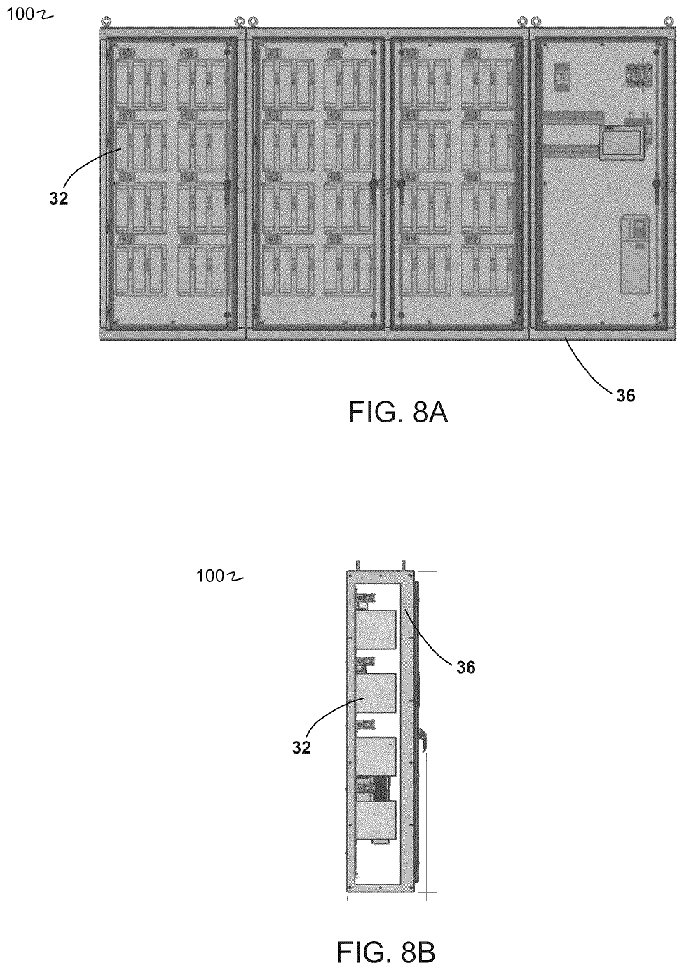

In alternative implementations, such as shown in FIGS. 7, 8A, and 8B, the ballasts 32 may be mounted in a cabinet 36, which may optionally be mounted remotely from the housing 10. As would be understood, mounting the ballasts 32 in a remote cabinet 36 may help to reduce the ambient temperature experienced by the ballasts 32, as the cabinet 36 may be installed in an area with a cooler environmental temperature of than the area where the housing 10 is installed.

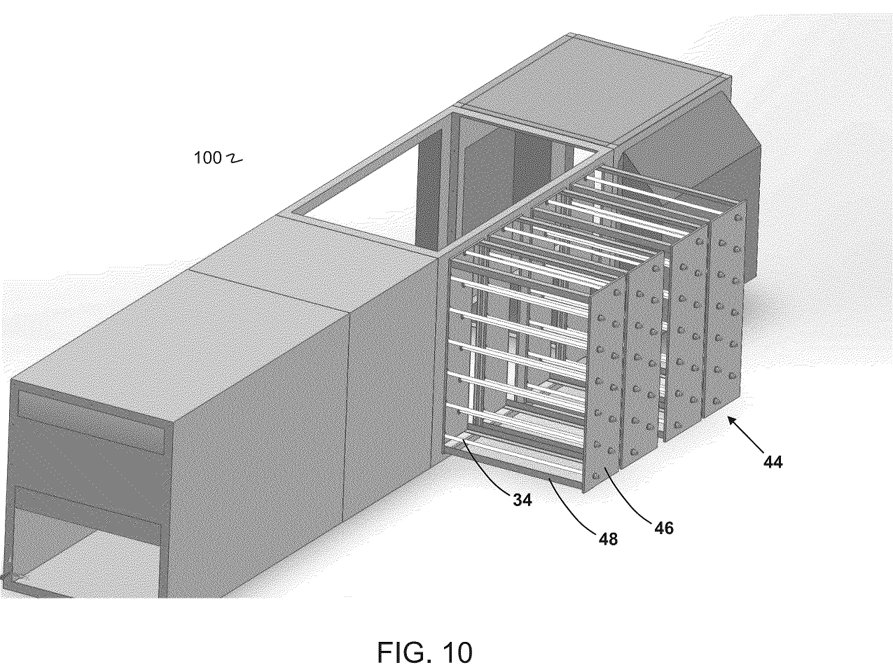

In some implementations, shown in FIGS. 5, 9 and 10, the lamps 34 are mounted in a plurality of cassettes 44. In various implementations, the system 100 includes two cassettes 44, three cassettes 44, four cassettes 44, or more. In certain implementations, the cassettes 44 are made from vertical plates 46 on which the lamps 34 are mounted and one or more cross bars 48 connecting the vertical plates 46 in a substantially perpendicular orientation. That is, the cassettes 44 are assembled to create a generally rectangular assembly with the lamps 34 spanning the length of the cassette 44, although alternative shapes and assemblies are possible and would be recognized by those of skill in the art.

A cassette 44, according to one implementation, may be seen in FIG. 9. In certain implementations, one of the vertical plates 46 is slightly larger than the opposing vertical plate 46, such that when the cassette 44 is inserted into the housing 10, the larger vertical plate 46 overlaps and seals against the opening in the housing 10 in which the cassette 44 is inserted.

In various implementations, the vertical plates 46 have a plurality of mounts 50 that are configured to hold one or more ends of the lamps 34, such that the lamps 34 are supported by the mounts 50 and the vertical plates 46. In some implementations, the mounts 50 are configured to allow electrical connectivity between the lamps 34 and the ballasts 32 when the lamps are mounted in the mounts 50. In such implementations, the mounts 50 are electrically connected to the ballasts 32 and possess a conductive element that allows electrical current from the ballasts 32 to pass through the mounts 50 into the lamps 34. In some implementations, the mounts 50 are formed from or possess a flexible polymer material that creates an air-tight seal with the cassette 44. According to some implementations, the flexible polymer material may be EPDM, PTFE, Buna-N, SBR, polyethylene, polypropylene, FKM, or any other polymer, material, or combination of materials that would be considered viable as an alternative in the art.

Turning back to FIG. 5, in some implementations, the cabinet 36 has doors 38. When the doors 38 are opened, the cassettes 44 may be removed from the housing 10 through the cabinet 36 to allow for access to the lamps 34 for cleaning, repair, or replacement. Further, when the doors 38 are closed the cassettes 44 are secured within the housing 10/cabinet 36 and are not able to be removed.

In various implementations, the fluid passing through the channel 16, especially when passing through the portion of the channel 16 within the primary reaction chamber 30, is permeated by the light emitted by the lamps 34. In such implementations, the light emitted may be UV light, and the light may directly contact contaminants in the fluid.

In various implementations, the contaminants may be, but are not limited to, volatile organic compounds (VOCs) which may break down when the light directly contacts them.

In some implementations, the fluid may contain molecular oxygen (O₂). In such implementations, when the light contacts the molecular oxygen in the fluid, the light may break the O₂ molecule into two independent oxygen atoms. These oxygen atoms may then react with other nearby O₂ molecules to produce ozone. The produced ozone molecules may then react with contaminants in the fluid, causing breakdown through oxidation. In some implementations, the light emitted from the lamps 34 breaks down contaminants in the fluid through both direct UV contact and ozone oxidation.

In various implementations, ozone formed in the primary reaction chamber 30 flows with the fluid passing through the channel 16 before reacting with other compounds, such as contaminants in the fluid.

As would be understood, ozone, in excessive concentrations, may be harmful to humans, animals, the environment, and objects prone to oxidation. Returning to FIG. 1, some implementations of the system 100 may reduce the risk of ozone escaping the housing 10 by including a secondary reaction chamber 52 within the housing 10. In these and other implementations, the secondary reaction chamber 52 is in sequence with the primary reaction chamber 30. In various implementations, the secondary reaction chamber 52 may possess a plurality of baffles 56, which are arranged within the secondary reaction chamber 52 to cause the channel 16 to take a more winding/longer path. As would be understood, the channel 16 with baffles 56 increases the total length of the channel 16 and increases the amount of time fluid passing through the channel 16 spends within the housing 10. Some implementations use a secondary reaction chamber 52 with baffles 56 to retain ozone contained in the fluid within the housing 10 until the ozone has fallen below a threshold value, such as a regulatory threshold for ozone emissions or a lower detection limit.

In some implementations, there is a temperature probe 58 positioned to measure the temperature of the fluid in the channel 16 as it leaves the primary reaction chamber 30 and enters the secondary reaction chamber 52. In various implementations, the temperature probe 58 is configured in relation to the PLC 20 and other devices in the same manner as the temperature probe 18 in the inlet duct 12 and temperature probe 28 in the tempering chamber 26 described above.

In various implementations, the PLC 20 will monitor the temperature of the fluid as detected by the temperature probe 58. In some implementations, if the temperature as measured by the temperature probe 58 has increased above a threshold value, determined by the temperature ratings of the lamps 34 or other equipment, the PLC 20 will send an output signal to the ballasts 32 to cease sending electrical current to the lamps 34. Various alternative devices and systems may be implemented to cool the fluid if the temperature exceeds the threshold temperature for operation.

Still referring to FIG. 1, in various implementations, an ozone monitor 60 may be mounted in, on, or around the housing 10, optionally near the outlet duct 14. The ozone monitor 60 may be in electronic communication with the PLC 20. The ozone monitor 60 measures the levels of ozone in the fluid passing through the channel 16. Measurements of ozone in the fluid near the outlet duct 14 are indicative of ozone levels that will leave the housing 10 into the outside environment. The PLC 20 may be configured to send commands to the ballasts 32 to adjust the electrical power sent to the lamps 34 in response to ozone levels measured by the ozone monitor 60 and reported to the PLC 20. In various configurations, if the ozone monitor 60 measures ozone levels that are too high for a particular application, such as above an upper threshold, the PLC 20 will command less power to the lamps 34. As would be appreciated, by reducing the power of the lamps 34 the amount of ozone produced in the primary reaction chamber 30 will be reduced. In other configurations, if the ozone monitor 60 measures ozone levels that are sufficiently low for a particular application such as below a lower threshold, the PLC 20 will command more power to the lamps 34 to increase cleaning efficacy. Various alternative mechanisms such as slowing airflow may be implemented to decrease ozone concentrations at the exit of the housing 10.

In some implementations, the system 100 may include an airflow sensor 62. The airflow sensor 62 may be configured to measure fluid flow through the channel 16. The airflow sensor 62 is optionally mounted within the cabinet 36, but may be mounted in a variety of locations as would be understood in the art. The airflow sensor 62 may be a differential pressure sensor, a turbine flow sensor, a vortex flow meter, a cup anemometer, a vane anemometer, a hot-wire anemometer, or any other sensor or meter that would be understood in the art.

In various implementations, the airflow sensor 62 is in electronic communication with the PLC. In some implementations, if the airflow sensor 62 measures fluid flow through the channel 16 that is sufficiently low, the PLC will send an output signal to the ballasts 32 to shut off electrical power to the lamps 34, such that ozone generation will cease if airflow is stopped or sufficiently slowed.

In various implementations, one or more gas detection probes 64 may be mounted in the housing 10. These gas detection probes 64 may be configured to detect a variety of products, byproducts, pollutants, or contaminants, as may be required by the application. These gases could include, but are not limited to, polycyclic aromatic hydrocarbons, nitrated polycyclic aromatic hydrocarbons, aldehydes, aromatic amines, nitrogen oxides, carbon monoxide, carbon dioxide, sulfur oxides, methane, ammonia, and various other gaseous compounds. In certain implementations, the gas detection probes 64 are in electronic communication with the PLC 20 to allow for reporting and data recording, as would be appreciated.

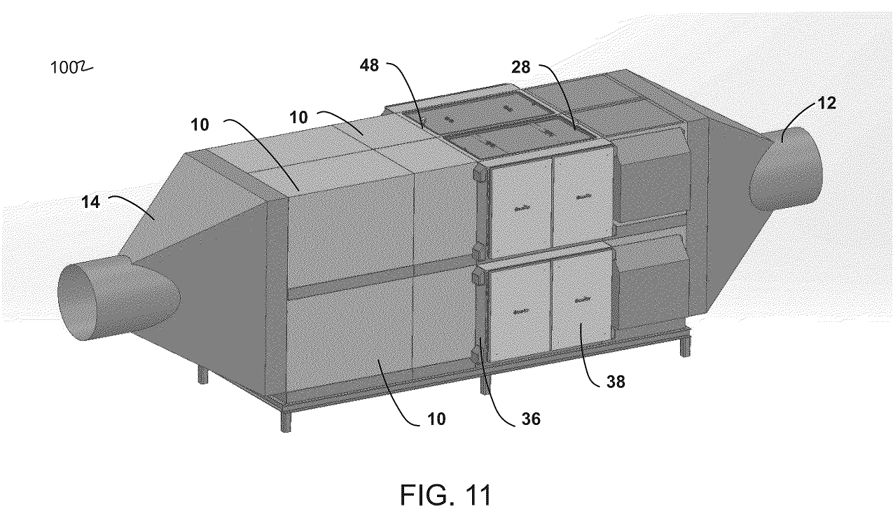

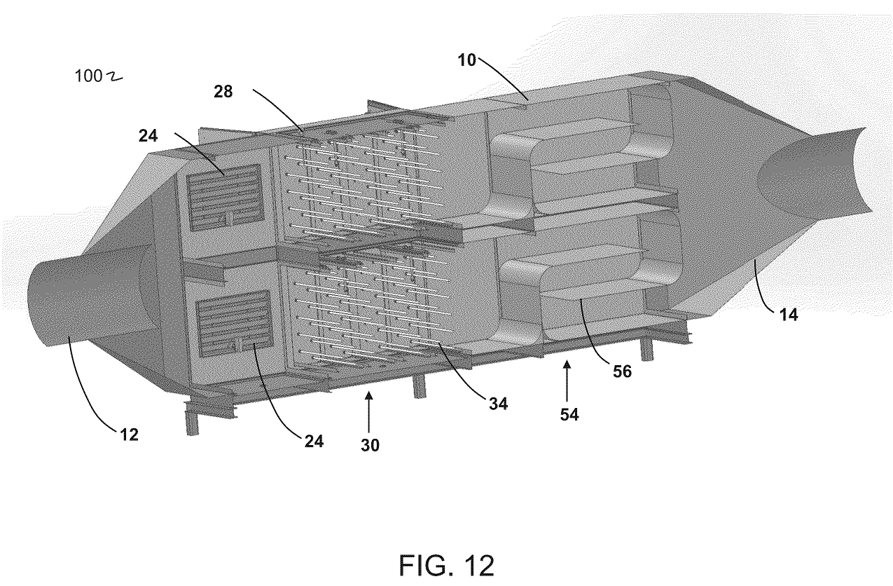

Turning to FIG. 11, in various implementations, the system 100 may have a plurality of housings 10 in operation in tandem. The various components discussed associated with each housing 10 may be included or omitted as needed and understood in the art. A cutaway of the system 100 with a plurality of housings 10 is shown in FIG. 12, as would be present in some implementations.

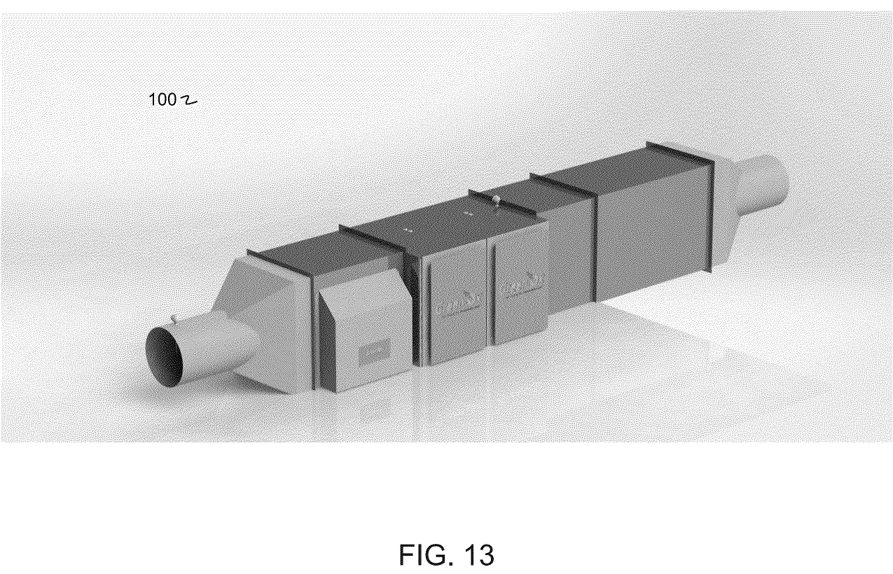

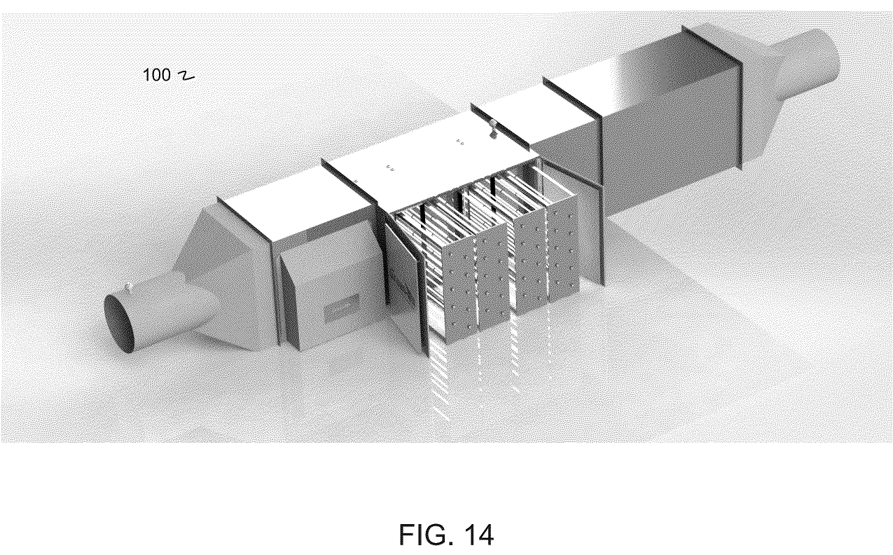

FIGS. 13 and 14 show the system 100 exterior with a singular housing 10, with cassettes inserted and removed respectively.

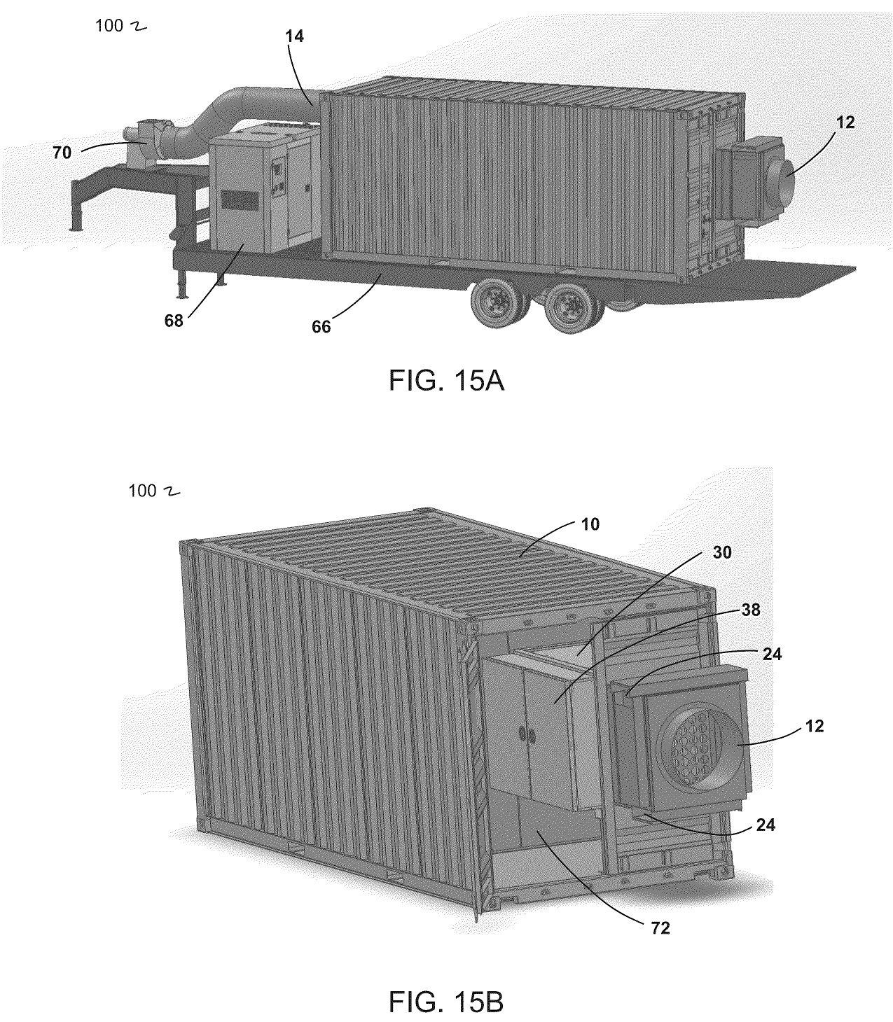

Turning now to FIG. 15A, in some implementations, the system 100 and housing 10 may be mounted onto a trailer 66 to allow the system 100 to be mobile.

In some implementations, the PLC 20 and other control equipment is housed in a control cabinet 68, optionally physically separate from the housing 10 and optionally mounted on the trailer 66. The control cabinet 68 may optionally have a human-machine-interface (HMI) located on its exterior or interior for human interaction with the system 100 such as inputting various temperatures, controls, or other values. The HMI may also allow for system overrides, manual control, and other features as would be understood.

In various implementations, the PLC 20, ballasts 32, and other electronic devices are rated at about 15% above their voltage and current requirements. The system 100 may also possess fan 70 to move the fluid through the channel 16.

In various implementations, the control cabinet 68, as well as other locations of the system 100, may have a main power disconnect that allows for disconnecting all power to the system 100 from external power sources, as well as the optional application of a safety lock to ensure power stays disconnected. The control cabinet 68, as well as other locations of the system 100, may have various emergency stops. These emergency stops may function to disconnect power to certain equipment, disconnect power to all equipment, or begin a particular shutdown sequence that is programmed into the PLC 20.

In some implementations, the system 100 may implement a fire alarm management system. The fire alarm management system may use various fire detection methods known in the art, such as ionization smoke detectors, photoelectric smoke detectors, thermistor heat detectors, electromagnetic flame detectors, or other equivalent fire detection methods. The fire alarm management system may use various devices and methods for fire suppression, such as the use of dry chemical fire suppressants, sprinklers, carbon dioxide, foam fire suppressants, and other fire suppression methods known in the art. Various implementations include the deployment of the fire suppression methods inside the housing 10 or outside of the housing 10, depending on the application and local requirements.

In some implementations, such as those shown in FIGS. 15A and 15B, the housing 10 may be constructed from an intermodal container or equivalent vessel. In such implementations, the housing 10 may contain an isolation barrier 72 that creates an air-tight barrier between two sections of the housing 10.

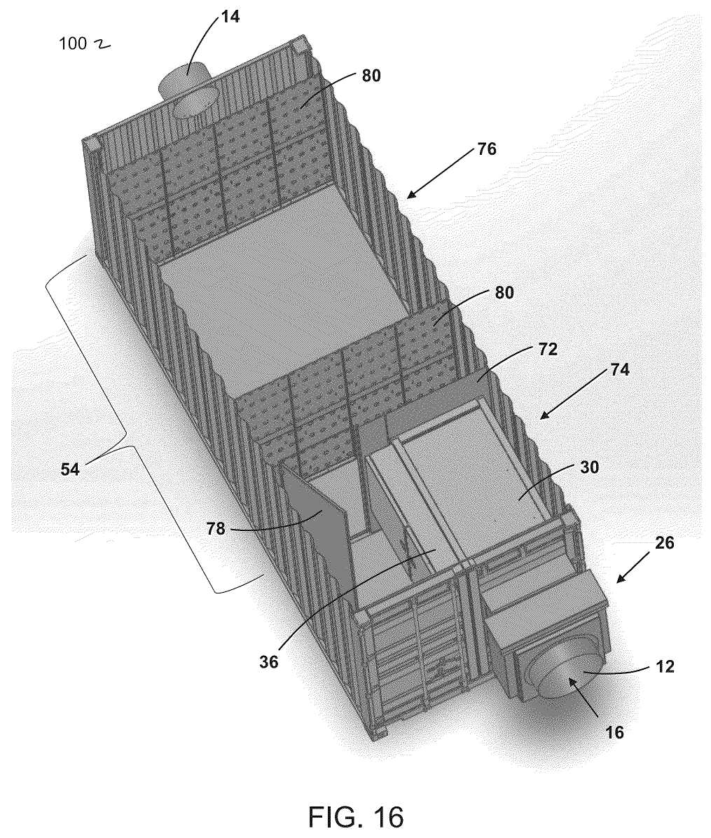

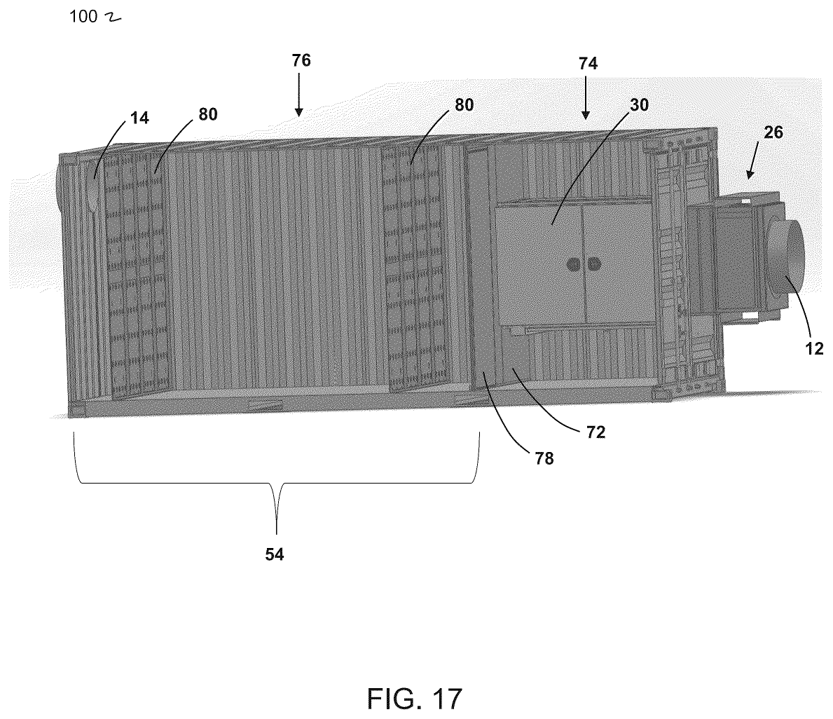

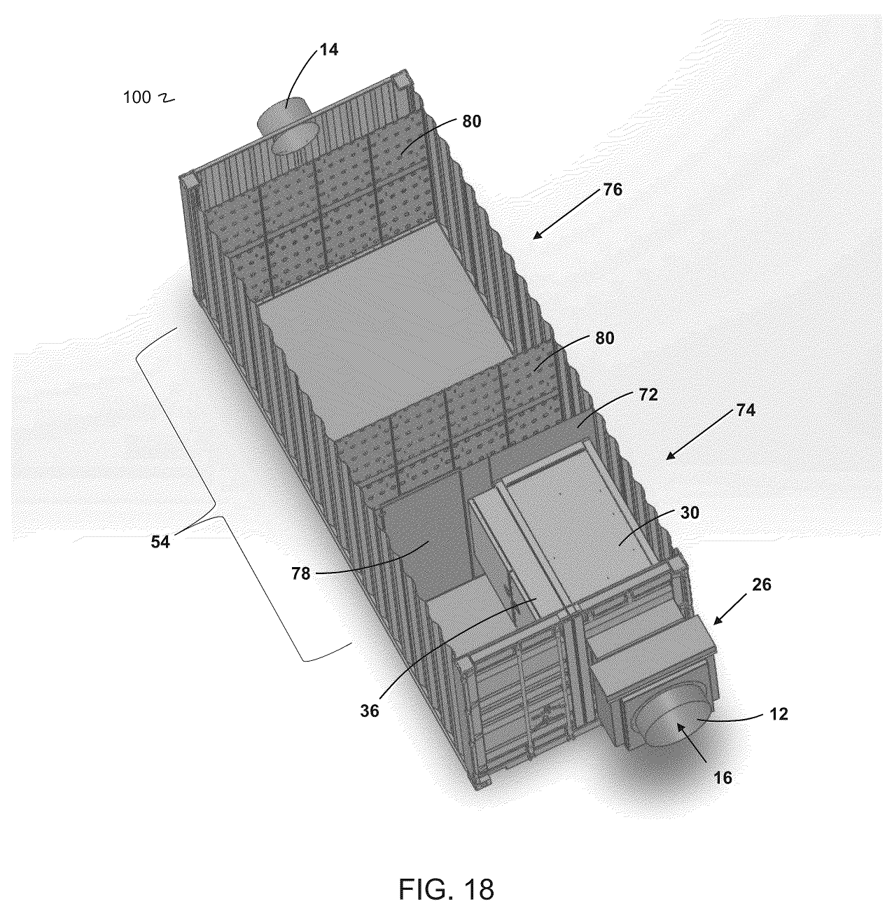

As best seen in FIGS. 16 and 17, the isolation barrier 72 separates the housing 10 into an occupied zone 74 and an unoccupied zone 76. The isolation barrier 72 may also have an access door 78 that allows for personnel to enter the unoccupied zone 76 while the system 100 is not in use. In some implementations, the secondary reaction chamber 54 occupies substantially the same space as the unoccupied zone 76. In various implementations, the purpose of the isolation barrier 72 is to prevent the entry of ozone in the secondary reaction chamber 54 to the occupied zone 74. FIG. 18 shows one implementation of the system 100 with access door 78 closed, as could be seen while the system 100 is in operation.

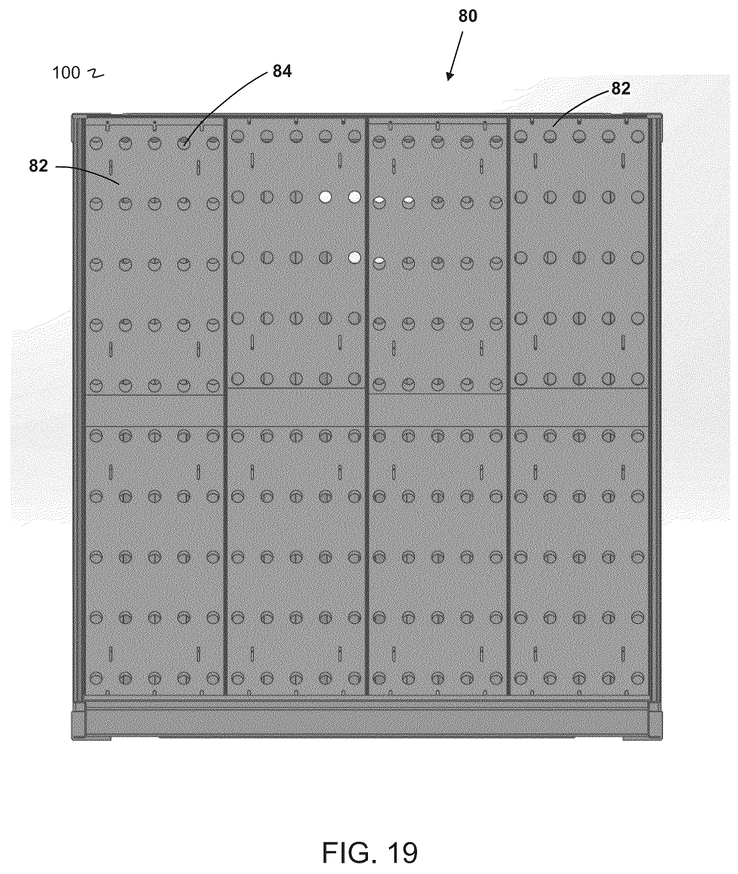

In some implementations, the secondary reaction chamber 54 does not include baffles. Optionally, the secondary reaction chamber 54 has one or more adjustable flow promoters 80. As best seen in FIG. 19, the adjustable flow promoters 80 consist of one or more stationary plates 82 that optionally collectively span the full cross-section of the housing 10 and one or more actuated plates 84 that are slidably affixed to the stationary plates 82. Both the stationary plates 82 and the actuated plates 84 have a plurality of openings. In some implementations, these openings are sized and positioned such that as the stationary plates 82 and actuated plates 84 slide past each other, their respective openings are completely aligned at one end of travel and completely misaligned (such as to create a solid barrier) at the opposite end of travel, with various degrees of opening alignment occurring throughout the distance of travel. In various implementations, as opening alignment increases, the total area through which fluid is able to pass increases, and as opening alignment decreases, the total area through which fluid is able to pass decreases. The alignment and misalignment of the openings in the adjustable flow promoters 80 may be adjusted to either increase or decrease the fluid pressure drop across each adjustable flow promoter 80. It would be understood that a larger fluid pressure drop across each plate, which is typical of a higher degree of opening misalignment, would result in a reduced flowrate of the fluid, which would in turn result in a higher residence time of a representative sample of fluid within the secondary reaction chamber 54. Likewise, it would be understood that a smaller fluid pressure drop across each plate, which is typical of a higher degree of opening alignment (larger openings), would result in an increased flowrate of the fluid, which would in turn result in a lower residence time of a representative sample of fluid within the secondary reaction chamber 54.

In various implementations, the adjustable flow promoters 80, baffles 56, or other equivalent technology may be called a flow restrictor.

In various implementations, each actuated plate 84 may be actuated vertically or horizontally relative to the corresponding stationary plate 82 by a plate actuator 86, shown in FIG. 2. The plate actuators 86 may be electric linear actuators, pneumatic linear actuators, hydraulic linear actuators, any of the proceeding with spring-return function, or any other equivalent actuation technology that would be understood as equivalent in the art. In some implementations, the plate actuators 86 are in electronic communication with the PLC 20. In such implementations, the PLC 20 may use input signals from the ozone monitor 60 or the gas detection probe 64 to calculate the required position of the actuated plates 84 to ensure satisfactory elimination of ozone or other fluid components.

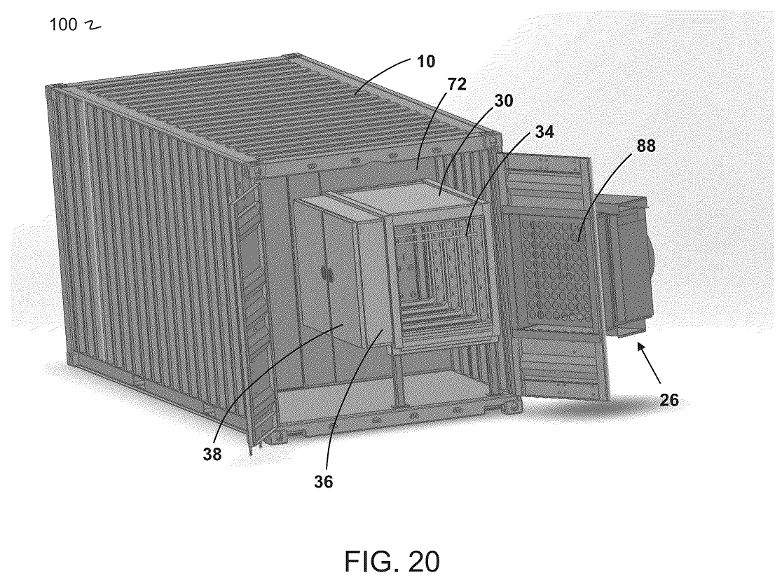

FIG. 20 shows one implementation of the system 100 with the housing 10 opened such that the tempering chamber 26 is separated from the primary reaction chamber 30. In some implementations, such as the one in FIG. 20, the tempering chamber 26 may have a perforated plate 88. In various implementations, the perforated plate 88 is a rigid sheet of material positioned in the tempering chamber 26 such that the perforated plate 88 intersects the entirety of the channel 16 passing through the tempering chamber 26. The perforated plate 88 may include a plurality of openings that improve flow characteristics of the fluid in the channel 16 to improve ozone generation and reaction rates.

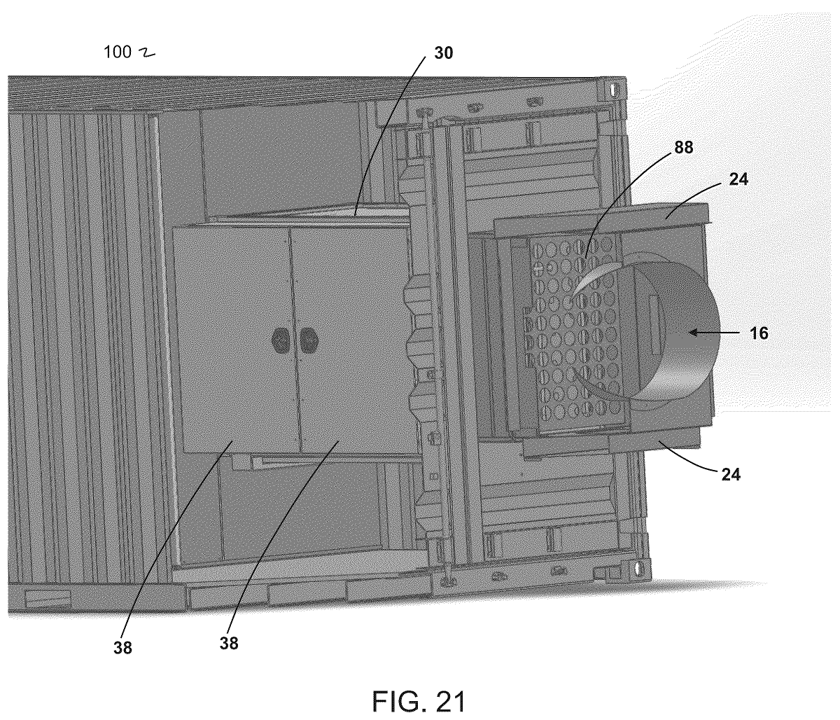

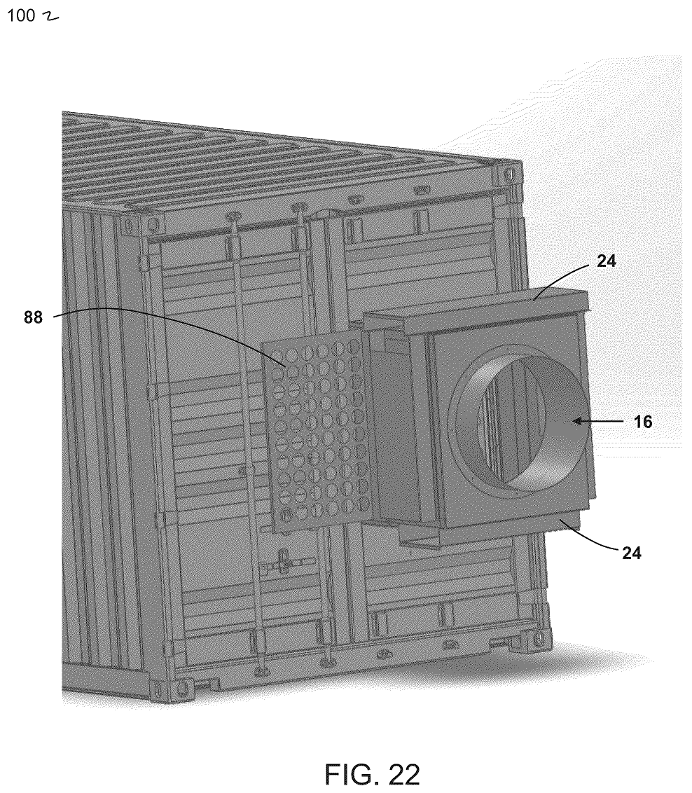

FIG. 21 shows a cut-away view of one implementation of the occupied zone 74 and tempering chamber 26. In various implementations, the perforated plate 88 is removable from the tempering chamber 26. As would be understood, the perforated plate 88 is removable, such as sliding the perforated plate 88 out of the tempering chamber 26 in a direction perpendicular to the channel 16, as shown in FIG. 22, or by equivalent methods.

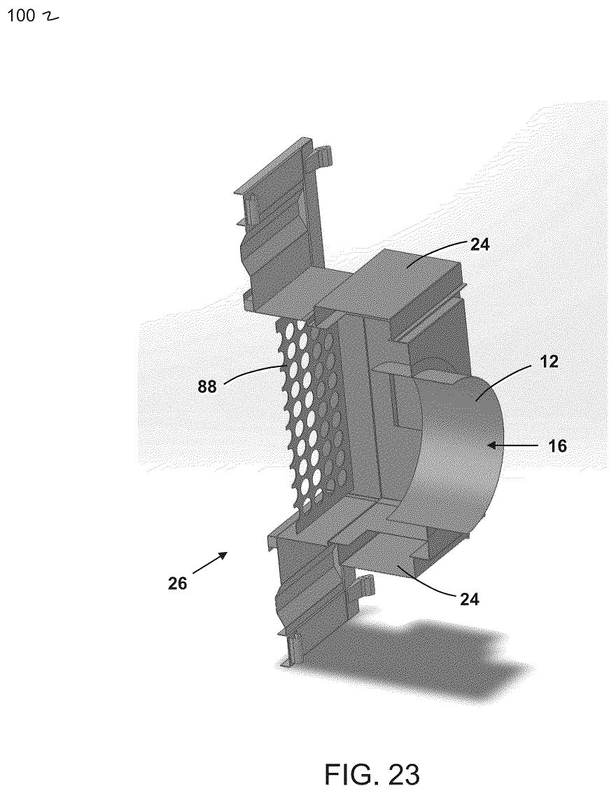

FIG. 23 shows a cut-away view of the tempering chamber 26 with the perforated plate 88 installed. In various implementations, such as those in FIG. 23, the inlet duct 12 may extend into the tempering chamber 26 such that the fluid passing through the inlet duct 12 will create a low-pressure zone through the Venturi effect at about where the makeup air dampers 24 are located. This low-pressure zone may then draw in fluid through the makeup air dampers 24 to reduce the overall fluid temperature.

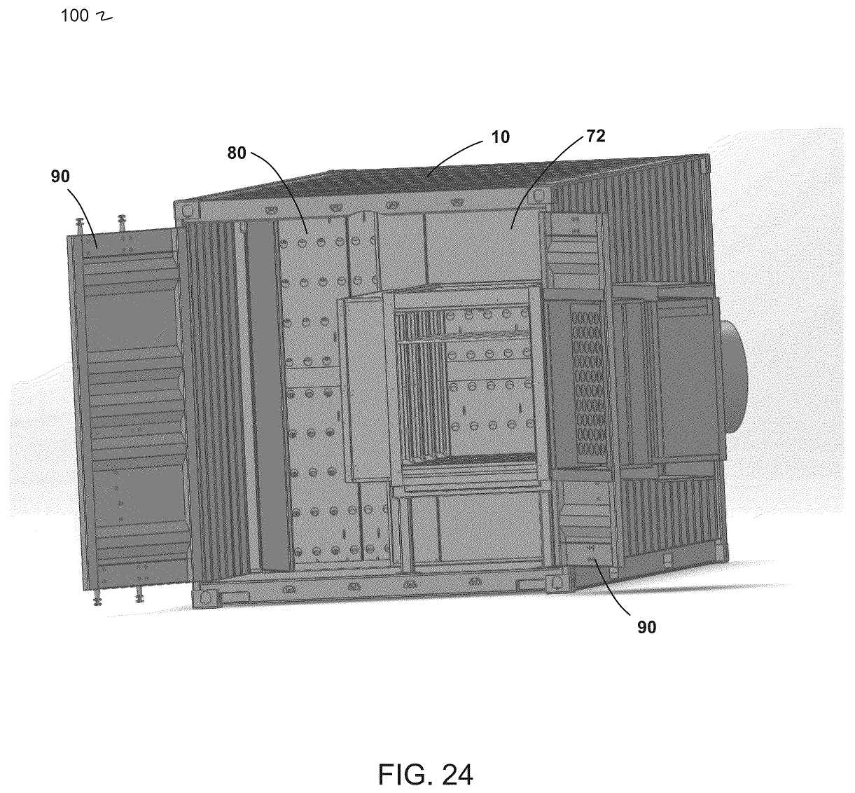

FIG. 24 shows one implementation of the system 100 in which the housing 10 includes one or more housing doors 90. The housing doors may be positioned at one end of the housing 10 and may occupy substantially all of the area of one side of the housing 10. The tempering chamber 26 may be mounted onto the housing doors 90. In some implementations, opening one or more housing doors 90 may provide access to the lamps 34 within the primary reaction chamber 30 for cleaning or maintenance. Likewise, in some implementations, opening one or more housing doors 90 may provide access to the tempering chamber 26 and perforated plate 88 for cleaning and maintenance. In further implementations, opening one or more housing doors 90 may provide personnel access to the occupied zone 74 of the system 100.

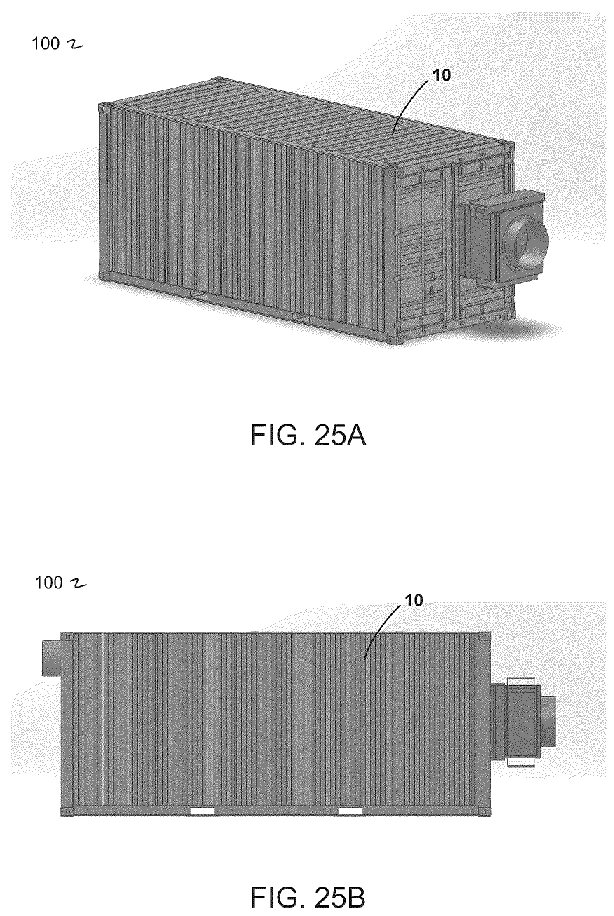

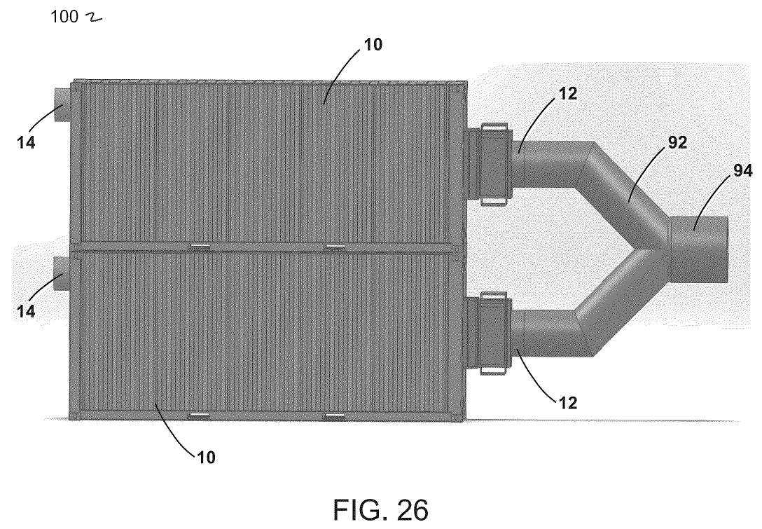

In some implementations, the system 100 may have one or more housings 10 functioning in tandem. FIGS. 25A and 25B show the system 100, according to some implementations, with a single housing 10 and accompanying components discussed above. FIG. 26 shows one implementation of the system 100 with two housings 10 where each housing 10 includes the same or substantially all of the same components. In this implementation, the housings 10 may be stacked allowing for space-efficient installations. In some implementations, several housings 10 may be fed from a single fluid source by connecting their inlet ducts 12 using a wye 92. The wye 92 may connect the several inlet ducts 12 of the system 100 to a common joint duct 94, which directs fluid from a space needing decontamination.

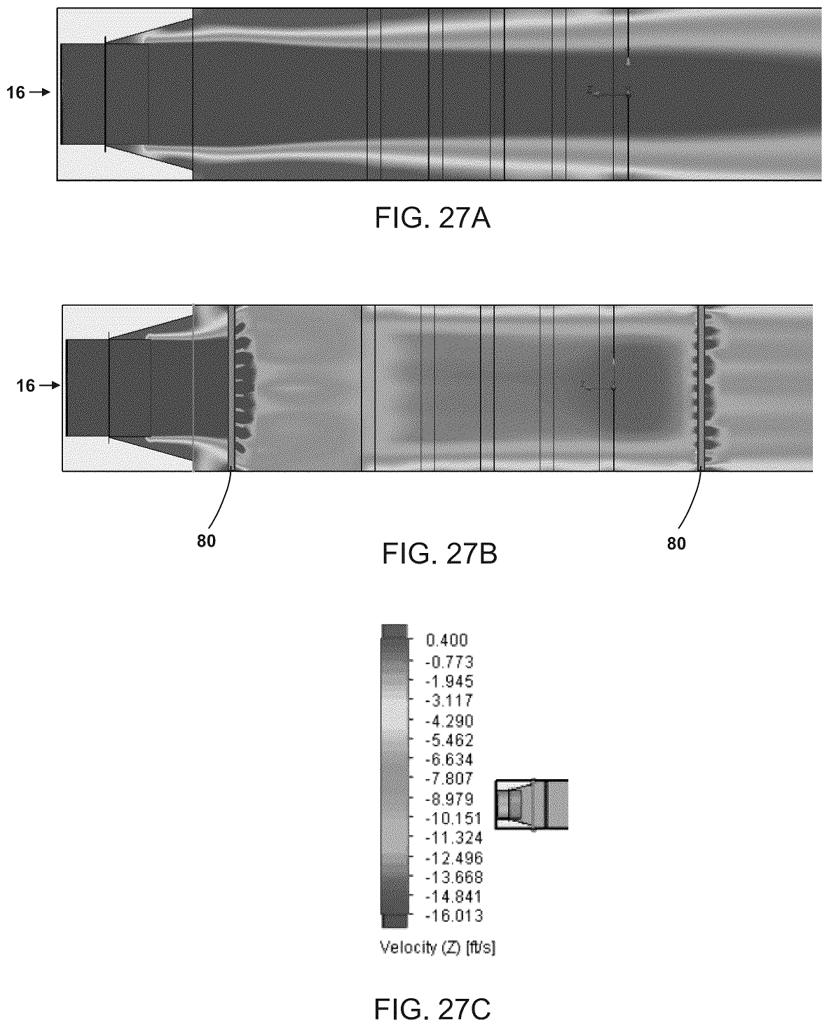

FIGS. 27A-27C show fluid velocity diagrams and accompanying scale of one implementation of the system 100. FIG. 27A shows the fluid passing through the channel 16 with no baffles 56 or adjustable flow promoters 80. FIG. 27C shows the scale used in FIG. 27A. As would be understood, referencing FIG. 27C in analyzing FIG. 27A shows that the fluid moving through the channel has a bulk velocity of about 16 feet per second. FIG. 27B shows, when analyzed alongside FIG. 27C, the fluid moving through the channel to have a bulk velocity of about 7.8 to 12.5 feet per second. As would be understood, a lower fluid velocity results in an increased residence time of any representative sample of fluid. This increase in residence time allows any remaining ozone within the fluid greater time to deplete.

Although the disclosure has been described with reference to preferred implementations, persons skilled in the art will recognize that changes may be made in form and detail without departing from the spirit and scope of the disclosed apparatus, systems, and methods.

What is claimed is:

1. A fluid purification system comprising:
(a) a housing open at each end through which a fluid is able to pass; and
(b) a cassette within the housing comprising a plurality of lamps capable of emitting light,
wherein the cassette is removable from the housing.

2. The system of claim 1, further comprising a plurality of ballasts capable of supplying electricity to the plurality of lamps.

3. The system of claim 1, wherein the lamps emit light in the ultraviolet wavelength band.

4. The system of claim 3, wherein the light in the ultraviolet wavelength band breaks down contaminants in the fluid.

5. The system of claim 3, wherein the light in the ultraviolet wavelength band generates ozone in the fluid.

6. The system of claim 5, further comprising an ozone monitor capable of detecting an ozone amount in the fluid, wherein the plurality of ballasts and plurality of lamps are configured to vary the amount of ultraviolet light emitted based on the ozone amount detected.

7. The system of claim 6, wherein a detected ozone amount in the fluid below a lower threshold causes the plurality of ballasts and plurality of lamps to increase the amount of ultraviolet light emitted and a detected ozone amount in the fluid above an upper threshold causes the plurality of ballasts and plurality of lamps to decrease the amount of ultraviolet light emitted.

8. A fluid purification system comprising:
(a) a housing with a first end and a second end through which a fluid is able to pass from the first end to the second end;
(b) a cassette within the housing comprising a plurality of lamps capable of spurring the generation of ozone;
(c) a backflow damper disposed within the housing at the first end of the housing capable of allowing, restricting, and stopping the passage of fluid through the housing; and
(d) a makeup air damper disposed proximal to the first end of the housing capable of allowing, restricting, and stopping the passage of fluid through the housing,
wherein the cassette is removable from the housing.

9. The system of claim 8, further comprising a plurality of ballasts capable of supplying electricity to the plurality of lamps.

10. The system of claim 9, further comprising an ozone monitor capable of detecting an ozone amount in the fluid.

11. The system of claim 8, further comprising an airflow sensor capable of measuring the flow of the fluid though the housing.

12. The system of claim 11, wherein the backflow damper is configured to close based upon the measured fluid flow by the airflow sensor.

13. The system of claim 8, further comprising a temperature probe positioned in the housing to measure the temperature of the fluid.

14. The system of claim 13, wherein the makeup air damper is configured to adjust its position based on the temperature of the fluid.

15. A fluid purification system comprising:
(a) a housing with a first end and a second end wherein a fluid is able to pass from the first end to the second end;
(b) a cassette within the housing comprising a plurality of lamps capable of spurring the generation of ozone; and
(c) a flow restrictor within the housing positioned between the cassette and the second end of the housing,
wherein the cassette is removable from the housing, and wherein the flow restrictor increases the time needed for fluid to pass through the housing.

16. The system of claim 15, wherein the flow restrictor is one or more adjustable flow promoters.

17. The system of claim 15, wherein the flow restrictor is a plurality of baffles.

18. The system of claim 15, wherein the lamps emit light in the ultraviolet wavelength band.

19. The system of claim 18, further comprising an ozone monitor capable of detecting ozone in the fluid.

20. The system of claim 19, wherein the lamps are configured to emit more or less ultraviolet wavelength light based on the ozone detected in the fluid.
